(19) United States
(12) Patent Application Publication
(10) Pub. No.: US 2004/0262418 A1
(43) Pub. Date: Dec. 30, 2004

(54) Air Freshener

(76) Inventors: Nigel Peter Smith, Devon (GB); John Daniel Robert Pickering, Devon (GB)

Correspondence Address: WELSH & KATZ, LTD, 120 S RIVERSIDE PLAZA, 22ND FLOOR, CHICAGO, IL, 60606, US

(21) Appl. No.: 10/492,673

(22) PCT Filed: Oct. 16, 2002
(86) PCT No.: PCT/GB02/04698

(30) Foreign Application Priority Data
Oct. 17, 2001 (GB) 0124987.9

(51) Int. Cl. A61L 9/04

(52) U.S. Cl. 239/43

(57) ABSTRACT

An air freshener comprises a liquid perfume (not shown) enclosed in a liquid tight container 1, with the container 1 comprising a perfume vapour permeable portion 3 so as to allow scented vapour to escape the container 1 whilst preventing any spillage or leakage of the liquid contained therein.

The present invention relates to an air freshener.

A number of different types of air freshener are known. A common feature of most air fresheners is the presence of a solid or liquid perfume that is sufficiently volatile under normal, or in some cases elevated, room temperatures that it will slowly vaporise, thereby providing a source of scented vapour over a period of time.

According to one aspect of the present invention, an air freshener is provided comprising a liquid perfume enclosed in a liquid tight container, the container comprising a perfume vapour permeable portion so as to allow scented vapour to emanate from the container whilst preventing any spillage or leakage of the liquid contained therein, wherein the container further comprises internal protrusions and/or indentations adapted to deflect the movement of the liquid inside the container when the container is agitated.

In a preferred embodiment, the air freshener further comprises a second liquid enclosed in the container, the second liquid being immiscible with the liquid perfume, and preferably denser than the liquid perfume and of a different colour. Optionally, both the first and, if present, second liquid may be thickened such that when the air freshener is agitated the resultant movement of the liquid inside the container is more viscous and occurs over an increased period of time.

According to another aspect of the present invention, an air freshener is provided comprising a liquid perfume enclosed in a liquid tight container, the container comprising a perfume vapour permeable portion so as to allow scented vapour to emanate from the container whilst preventing any spillage or leakage of the liquid contained therein, wherein said container contains two immiscible liquid phases of differing density, the liquid perfume constituting or being associated with one of the phases and being substantially immiscible with the other of said phases. Preferably at least one of the liquid phases is coloured. More preferably, each phase is of a different colour.

When the second liquid is denser than the liquid perfume, the latter is able to reach the vapour permeable membrane and thus escape into the ambient air when the container is at rest. If, on the other hand, the second liquid is less dense than the liquid perfume, some agitation of the container and its contents will be required to permit the fragrance to escape.

Preferably the second liquid is denser than the liquid perfume and more preferably is water-based. The second liquid may be water containing up to about 20% by weight (preferably 13% by weight) of an electrolyte, e.g. sodium chloride. It may also contain a water-soluble preservative.

The body of the container may be fabricated using a suitable grade of Surlyn^{R}; alternative materials eg glass may also be used. We have found that Surlyn^{R} grade PC2000 (a sodium-based ionomer) is well suited for this purpose because of its high clarity and mouldability.

Preferably the perfume vapour permeable portion comprises a gas permeable membrane comprising a polymer such as high density polyethylene (HDPE). Preferably, the gas permeable membrane is a Surlyn^{R}/polymer coextrudate or laminate. The Surlyn^{R} side of the coextrudate is preferably heat-sealed to the body of the container. We have found that this arrangement provides an excellent seal with the body of the container when the latter is itself formed of Surlyn^{R}.

Preferably the polymer is less than 120 microns in thickness, typically being from 10-80 microns, more preferably from 20-50 microns, most preferably 40 microns. The Surlyn^{R} layer of the coextrudate is preferably 5-25 microns, more preferably 5-15 microns, and most preferably 10-12 microns in thickness.

As an alternative to HDPE, the polymer could be low density polyethylene (LDPE) or medium density polyethylene (MDPE) or polypropylene (PP) or polyvinyl chloride (PVC) or ethylene vinyl acetate (EVA) or a mixture of two or more of HDPE, LDPE, MDPE, PP, PVC and EVA.

Advantageously, the container further comprises a gas impermeable membrane bonded to the gas permeable portion so as to prevent scent escaping the container before removal of the gas impermeable portion to activate the air freshener. Preferably the gas impermeable portion comprises a layer of foil. Such a foil is advantageously lightly bonded to the outer surface of the gas permeable membrane so as to prevent fragrance passing through the membrane and accumulating beneath the foil.

An air freshener in accordance with this invention can be constructed as a free-standing, decorative unit which generates interesting visual effects when the unit is agitated (these being caused by the mingling of the immiscible liquids).

In a preferred embodiment of the present invention, the air freshener comprises at least two liquids enclosed in a sealed container. The first liquid is a coloured liquid perfume formula which, under normal atmospheric conditions, will slowly vaporise so as to provide a scented vapour. The second liquid is more dense than the first, immiscible therewith, and of different colour. Furthermore, the container is preferably formed entirely of transparent material, such that when the container is at rest the two liquids are clearly visible as two differently coloured layers of liquid. This provides both an interesting visual effect, and a ready means for assessing the remaining life-span of the air freshener, as over time the volume of liquid perfume present in the container will decrease as it slowly vaporises.

In this preferred embodiment, the liquid perfume and second liquid are preferably present in the container initially so that the liquid perfume makes up from 40 to 75%, preferably about 50%, of the total liquid volume. The second liquid may suitably consist of water, while the liquid perfume may consist of neat fragrance, or a blend of fragrance and a suitably water-insoluble solvent such as Isopar (a blend of alkanes), the two liquids being coloured by suitable water-soluble and oil-soluble dyes. Alternatively the second liquid may be a silicone oil or IPM. The selection of suitable liquids will in general be made in accordance with their density, colour or readiness to adopt colour from a solute, and mutual immiscibility.

Advantageously the liquid-tight container is an injection moulded component. Conveniently the container has an internal volume of from 5 to 100 ml, preferably about 20 ml.

A specific embodiment of the invention will now be described, by way of example, with reference to the accompanying drawings, in which:

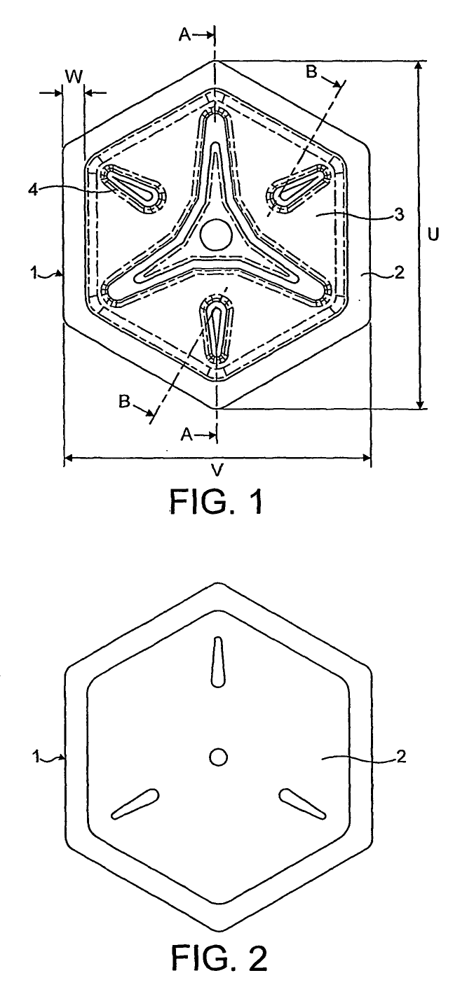

FIG. 1 is a top view of a container of an air freshener according to the present invention;

FIG. 2 is a bottom view of the container of FIG. 1;

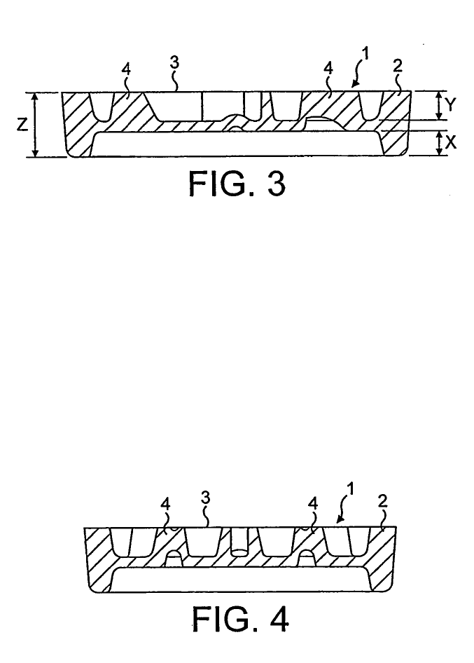

FIG. 3 is vertical section through the container of FIG. 1 along the line A-A; and

FIG. 4 is a vertical section through the container of FIG. 1 along the line B-B.

In the drawings, a container of an air freshener according to an embodiment of the present invention is generally shown.

Referring now to FIGS. 1 and 2 in particular, the container 1 comprises an open topped container body 2, somewhat similar in shape to an ashtray, and a container lid in the form of a membrane 3 that closes off the container body 2. The membrane 3 is sealed to the rim of the container body 2, so as prevent leakage or spillage of the liquids within the container 1, but is made of material that is gas permeable at room temperature, so as to allow vaporised perfume to escape the container 1.

The container body 2 consists of Surlyn^{R} Grade PC2000 (DuPont Packaging & Industrial Polymers, PO Box 50, CH-1218 Le Grand-Saconnex), which is the trade name for a range of ionomers (ionically cross linked polymers), and is formed by injection moulding. Grade PC2000 is particularly suitable for use as the container body in this invention due to its chemical inertness, high clarity and moldablity.

The membrane 3 is made from a Surlyn^{R}/high density polyethylene coextrudate, with the Surlyn^{R} being Grade PC100 and being heat sealed to the container body 2. The Surlyn^{R} and HDPE may be co-extensive. Alternatively the Surlyn^{R} component maybe annular so as to provide an area of contact the body of the container while leaving a thinner central area of HDPE alone which will this have a higher vapour permeability than a laminate or coextrudate consisting of co-extensive layers. In this embodiment, the Surlyn^{R} layer is 10 microns in thickness and the HDPE layer is 40 microns in thickness.

Optionally, the air freshener may be supplied with a gas impermeable membrane (not shown), such as a foil layer, lightly bonded to the outer surface of the gas permeable membrane 3, so as to prolong the shelf-life of the air freshener before it is desired to be used. The bonding of the foil layer to the gas permeable membrane 3 effectively prevents any vaporised perfume from escaping the container 1 or building up between the foil and membrane 3, but the foil layer should be removable with relatively little effort so as to allow easy activation of the air freshener.

Referring now to FIGS. 3 and 4 in particular, the inner surface of the container is provided with a number of protrusions 4 so as to provide a series of weirs or deflectors. The gas permeable membrane 3 is bonded to the tops of these projections as well as to the rim of the container body 2. Thus, on shaking or inversion of the container the two liquids flow around the protrusions, causing the liquids to intermingle momentarily before settling again into two layers and providing an interesting visual effect. Such agitation may also result in a momentary increase in vapour production and thus a “burst” of scent.

With reference to FIGS. 1 and 3, suitable dimensions for the air freshener are as follows: an overall container depth of 17 mm (dimension Z); an internal container depth of 7.5 mm (dimension Y); a container base depth of 7 mm (dimensionX); a container rim width of 5.5 mm (dimension W); and overall container width in a first direction of 78.21 mm (dimension V); an overall container width in a second direction of 89.65 mm (dimension U); a liquid perfume volume of initially 10 ml (not shown); and a second liquid volume of 10 ml (not shown).

1. An air freshener comprising a liquid perfume enclosed in a liquid tight container, the container comprising a perfume vapour permeable portion so as to allow scented vapour to escape the container whilst preventing any spillage or leakage of the liquid contained therein, wherein the container further comprises internal protrusions and/or indentations adapted to deflect the movement of the liquid inside the container when the container is agitated.

2. An air freshener as claimed in claim 1, wherein the liquid perfume is coloured.

3. An air freshener as claimed in claim 1, wherein the air freshener further comprises a second liquid enclosed in the container, the second liquid being immiscible with the liquid perfume, and of different density and colour.

4. An air freshener as claimed in claim 1, wherein the liquid perfume and, if present, second liquid are thickened such that when the air freshener is agitated the resultant movement of the liquid inside the container is viscous.

5. An air freshener as claimed in claim 1, wherein the perfume vapour permeable portion comprises a gas permeable membrane comprising a polymer.

6. An air freshener as claimed in claim 5, wherein the gas permeable membrane is a Surlyn^{R}/polymer coextrudate.

7. An air freshener as claimed in claim 5, wherein the body of the container also comprises Surlyn^{R}.

8. An air freshener as claimed in claim 1, wherein the container further comprises a gas impermeable membrane bonded to the gas permeable portion so as to prevent scent escaping the container before removal of the gas impermeable portion so as to activate the air freshener.

9. An air freshener as claimed in claim 8, wherein the gas impermeable membrane comprises a layer of foil.

10. An air freshener comprising a liquid perfume enclosed in a liquid tight container, the container comprising a perfume vapour permeable portion so as to allow scented vapour to emanate from the container whilst preventing any spillage or leakage of the liquid contained therein, wherein said container contains two immiscible liquid phases of differing density, the liquid perfume constituting or being associated with one of the phases and being substantially immiscible with the other of said phases.

11. An air freshener as claimed in claim 10, wherein the phase associated with said liquid perfume is less dense than the other of said two immiscible liquid phases.

12. An air freshener as claimed in claim 10, wherein the perfume vapour permeable portion comprises a gas permeable membrane comprising a polymer.

13. An air freshener as claimed in claim 12, wherein the gas permeable membrane is a Surlyn^{R}/polymer coextrudate.

14. An air freshener as claimed in claim 12, wherein the body of the container is fabricated using Surlyn^{R}.

15. An air freshener as claimed in cliam 10, wherein the container further comprises a gas impermeable membrane bonded to the gas.
